(19) United States
(12) Patent Application Publication
(10) Pub. No.: US 2004/0158236 A1
(43) Pub. Date: Aug. 12, 2004

(54) Surgical needle with laser target

(76) Inventor: Reinhardt Thyzel, Gommiswald (CH)

Correspondence Address: Lloyd McAulay, Reed Smith LLP, 599 Lexington Avenue, New York, NY, 10022-7650, US

(21) Appl. No.: 10/364,880

(22) Filed: Feb. 12, 2003

(51) Int. Cl. A61B 18/20

(52) U.S. Cl. 606/16

(57) ABSTRACT

A surgical needle for fracturing tissue such as cataracts has a distal operating port which holds tissue to be fractured. An optical fiber that extends down a needle applies laser energy pulses to a target causing optical breakdown and the generation of shockwaves which impinge on the tissue at the operating port, causing the tissue to fracture. Fractured tissue is aspirated at the passageway of the surgical needle. The operating port and target are both positioned at the distal end of the needle to facilitate surgeon observation during the operation. The needle has a wall which is unitary and provides an aspirating channel with a smooth surface so as to minimize flow turbulence and maximize laminar flow. The combination of laminar flow results in greater flow velocity and thus enhanced ability to hold tissue at the port.

### BACKGROUND OF THE INVENTION

In general, this invention relates to a laser powered surgical instrument which provides shockwaves for the ablation of tissue and more particularly to one that provides certain improvements over the surgical instruments shown in U.S. Pat. No. 5,324,282 and No. 5,906,611.

The embodiment of the invention described is adapted to be used in eye surgery and particularly for cataract removal. However, the invention can be embodied in devices which are adapted to other surgical purposes.

The use of laser energy for eye surgery is well known. More particularly, employment of laser energy directed to a metal target to generate shockwaves which impinge on tissue to break up the tissue is known in the above referenced two patents.

The primary purpose of this surgical needle is for cataract surgery. The cataract tissue is held at the distal opening of the needle and is broken up by shockwaves that shatter the tissue on which the shockwaves impinge. These shockwaves are generated by application of laser pulses on a metal target located within the surgical needle adjacent to the opening of the needle at which the targeted tissue is positioned.

The surgical needle designs shown in the above two referenced patents have been successfully employed in operations; the stepped target design of the '611 design being preferred.

However, there are operating features of known operating needles which it is desirable to improve and that would provide an enhanced surgical instrument.

More particularly, it is desirable that the device permits completion of the procedure with less operating time and use of less energy.

One advantage of a shorter operating time is that it can provide less trauma and less risk to the patient. This enhanced patient function occurs only if the shorter operating time is accompanied by such operating parameters as requiring less energy and providing an enhanced ability for the surgeon to navigate the needle with assurance of position.

For example, one feature that the surgeon refers to as “occlusion” is the ability of the distal opening to hold the tissue in place as it is being shattered by the shockwaves. In large part because it aids in providing a shorter operating time, it is an object of this invention to enhance the occlusion. When cataract tissue has been broken off by a pulse of energy, it is frequently too large to aspirate out of the small aspirating passageway in a needle. It is important that the tissue be held at the distal operating port of a needle so that a second or third pulse of ultrasonic energy will break down the tissue for ultimate aspiration.

A greater flow velocity of aspirating fluid will help to rapidly remove fractured tissue so that the ablating of tissue can proceed without obstruction and thus more rapidly. To achieve this greater velocity of aspirating fluid, it is desirable that there be as little turbulence as possible. Flow that is close to the laminar flow will permit a more rapid flow of fluid and thus a more rapid removal of fractured or ablated tissue. A greater flow velocity will create a greater vacuum at the operating port that better holds the tissue and provides enhanced occlusion.

Thus it is an object of this invention to provide a structure and technique that provides enhanced occlusion and greater flow velocity.

It is important that the above objects be obtained in a device where additional structural features or complicated procedures are not required so that costs can be minimized and the surgeon will feel as comfortable as possible in using the device and the procedure associated therewith.

### BRIEF DESCRIPTION

In brief, one embodiment of this invention involves a 1.2 mm outside diameter needle having a distal operating port of about 0.6 mm to 0.8 mm. The operating port of the needle is at the distal end of the needle so that the surgeon's view of the operating area is minimally blocked. The target on which the laser energy impinges to generate the acoustic shockwaves is adjacent to the distal end of the target and positioned close to the operating port.

By having a shorter operating time, the target can be somewhat less massive than in the prior art. Thus the needle is designed with a blunt end at which the operating port is positioned so that the operating area can be more readily observed.

The 20.5 mm long needle is made of a unitary metal without seams. This lack of seams reduces turbulence and permits laminar flow or laminar-like flow than in the prior previous designs thereby quickly removing fractured tissue and also providing a flow velocity that better holds the tissue at the operating port. This contributes to a lesser operating time.

The target structure is best understood by reviewing the drawings. It is a structure somewhat different than the stepped target shown in U.S. Pat. No. 5,906,611. The structure provides an optimum geometry to supply enough target material for the operation without requiring a more massive amount of target material. This less massive target makes possible an overall geometry which facilitates the use of this operating needle.

In large part, because of a greater flow velocity, a better occlusion of larger fragment tissue pieces is obtained at the operating port to assure immediate further shattering by subsequent shockwaves and then aspiration through the needle.

### ON TERMINOLOGY

It is worthwhile keeping in mind a distinction between flow velocity, flow rate and flow volume. In large part because of the smaller operating port, the amount of fluid that is aspirated in a given time period (i.e., flow rate) is reduced over the prior art design. But because of a greater aspirating vacuum, the velocity of the fluid being aspirated into and through the needle is greater. This greater flow velocity helps to increase the occlusion characteristic of the operating port. Further, even though the flow velocity in the needle is increased, a shorter operating time and lower flow rate means that the total volume of flow is reduced over the prior art. Thus, in this design, an increased flow velocity is coupled with a decreased flow rate and decreased flow volume.

### BRIEF DESCRIPTION OF THE FIGURES

FIG. 1 is a longitudinal sectional view through the first embodiment of the surgical needle of this invention.

FIG. 2 is an expanded sectional view of the distal end of the FIG. 1 instrument.

FIG. 3 is a side view of the distal end of the FIG. 1 instrument.

FIG. 4 is a perspective view of the distal end of the FIG. 1 instrument.

FIG. 5 is a partially cut away perspective view of the FIG. 1 distal end.

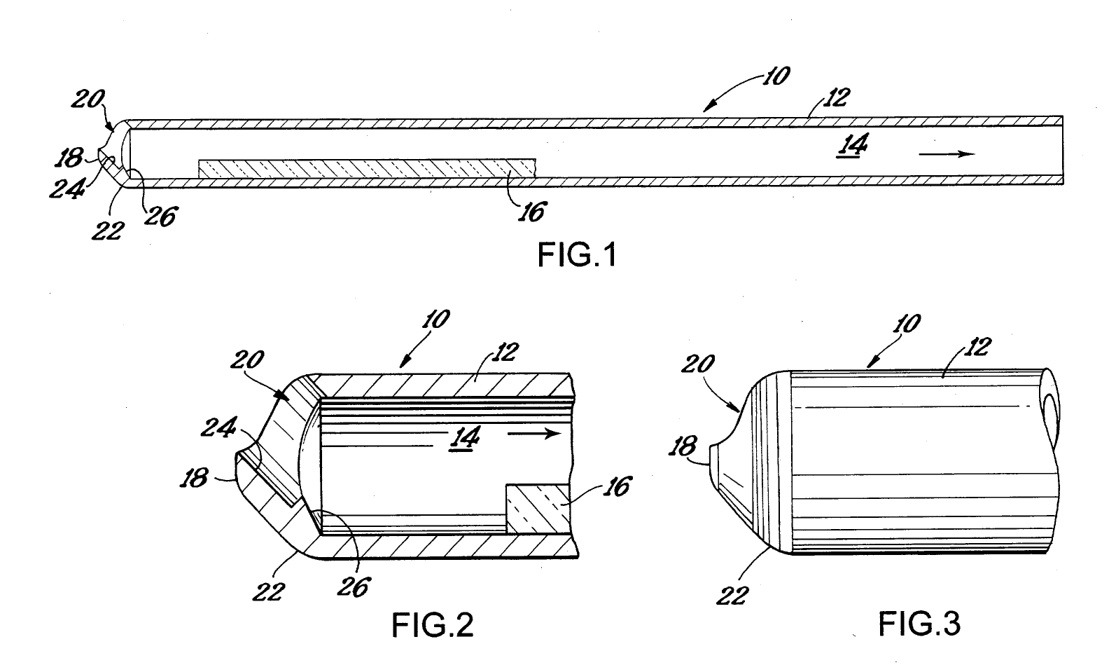

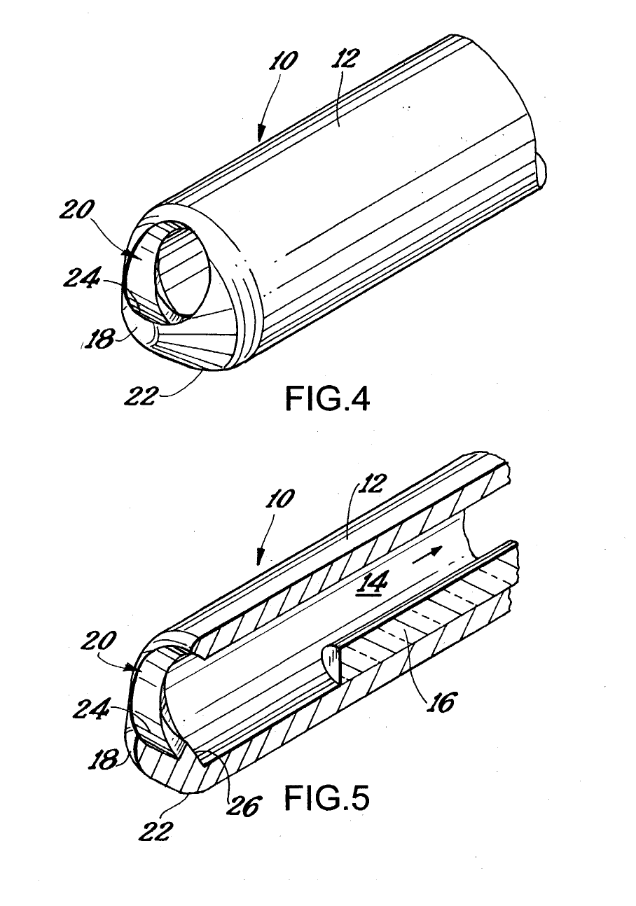

### DESCRIPTION OF THE PREFERRED EMBODIMENTS

As shown in the FIGs., all of which refer to the same embodiment, the surgical needle 10 has a unitary sidewall 12, an aspirating channel 14 and an optical fiber 16 adapted to convey laser energy.

The distal end of the needle 10 has a relatively blunt front surface 18 and an operating port 20. The target, 22 is a complex surface comprising a primary surface 24 and a small hill 26. The central axis of the optical fiber 16 is in alignment with the hill portion 26 so that when the optical fiber supplies pulses of laser energy, the central component of those pulses will impinge on the hill 26 causing optical breakdown and the release of shockwaves that are then transmitted to the operating port 20. After the hill 26 has been ablated away, the main portion of the laser pulse energy will impinge on the main surface 24 providing further shockwaves.

Because of the unitary sidewall 12, there are no ridges or breaks or discontinuities in the sidewall 12 which would induce turbulence. It is true that at the port 20, the suction of fluid into the aspirating channel 14 causes turbulence as does the front edge of the optical laser energy fiber 16. However, the smooth inner surface over ninety (90) percent of the needle which is proximal of the front end of the fiber 16 promotes a more laminar type of flow and thus permits a greater flow velocity than otherwise would be the case.

Irrigation is provided by a separate irrigating needle (not shown) of a type known in the art.

The circular port is preferable over an elliptical port. The reason is that for a given maximum size particle to be aspirated, the circular port has a lesser cross-sectional area and thus provides a better trade-off of higher flow velocity and lower flow volume.

This combination of structural features provides a more optimum trade-off of functional features. For example, a somewhat smaller needle 10 (1.2 mm outside diameter and 0.9 mm inside diameter) is tolerable because the flow velocity is enhanced. The flow velocity is enhanced because of the less turbulent more laminar flow. This less turbulent more laminar flow arises because of a structural design which includes the unitary needle wall 12 having a smooth inside surface. The higher velocity flow due to a less turbulent aspirating flow, permits the use of a lesser quantity of fluid to provide an enhanced aspirating effect that permits a smaller diameter needle.

In large part, because of the greater flow velocity, pieces of tissue that have been ablated are more readily held at the operating port 20 to be shattered into smaller pieces that can be more readily aspirated by immediately successive shockwave pulses. This enhanced occlusion results in a shorter operating time.

In part as a consequence of the shorter operating time, the target 22 need not be as massive as in previous designs. Thus it can be designed to permit a needle at which the operating port 20 is at the distal end, rather than requiring a set back to accommodate a more massive target. In one embodiment, the thickness of the target 22 over the main target surface is 0.21 mm.

Having the operating port 20 at the distal end means that the surgeon's view of the operating zone where the tissue ablation occurs is minimally obstructed by the front surface of the needle. This provides the surgeon with a greater ability to navigate the needle with assurance and precision thereby contributing to the shorter operating time.

It is presently believed that a somewhat shorter laser pulse length (for example, four nano-seconds) may be advantageous in reducing the mass of target required, thereby contributing to most of the other parameters discussed above, while delivering adequate energy shockwaves to ablate tissue particularly where the tissue particles are better occluded at the port 20 so that they can be more quickly disposed of as smaller aspirated pieces by immediate successive shockwaves.

As may be seen in the above description, this combination of features positively reinforce one another to provide an optimum design. In a sense, many of these features are not so much trade-offs with one another as features which make it possible for the other feature to be effective.

For example, less turbulent flow due to the unitary sidewall 12 provides better occlusion which makes it possible to reduce the operating time which therefore allows for a less massive target 22 which in turn permits the tip design in which the port 20 is at the distal end so that the surgeon can better navigate the needle thereby reducing operating time that in turn permits the reduced mass of the target.

With respect to the target 22, the hill 26 is created by the forming technique that creates the port 20. The small zone between the hill 26 and the surface 24 could be filled in and the device operate as intended.

The target 22 has some similarities to the stepped target in U.S. Pat. No. 5,906,611 except that the key target surface 24, and even the target surface of the hill 26, are at an angle (approximately 45 degrees) to the axis of the needle thereby providing a more direct path between the shockwaves generated in the mouth 20 than in the '611 patent design. It is believed that this more direct path makes a given energy shockwave more effective in breaking up tissue at the operating port 20.

In one preferred embodiment, the following dimensional arrangements exist. The needle 10 is 20.5 mm long, has an outside diameter of 1.2 mm, and an inside diameter of 0.9 mm and thus a very thin wall of 0.15 mm. The laser fiber is 0.34 mm in diameter. In that embodiment, the operating port 20 is circular and has a diameter of 0.6 mm to 0.8 mm and the axis of the port 20 is at approximately 45 degrees to the axis of the needle 10. The front end of the optical fiber 16 is 1.9 mm from the distal edge of the needle and approximately 1.3 mm from the beginning point of the operating surface provided by the hill 26. The curved distal tip of the needle 10 is approximately a spherical surface having a radius of curvature of 0.6 mm.

In that embodiment a known YAG laser provides laser energy at 1.064 nano-meters in pulses having about 4 nano-second widths. The needle 10 including the target 22 is titanium.

While the foregoing description and drawings represent the presently preferred embodiments of the invention, it should be understood that those skilled in the art will be able to make changes and modifications to those embodiments without departing from the teachings of the invention and the scope of the claims.

What is claimed is:

1. In a surgical needle for fracturing tissue at an operating port through the generation of shockwaves due to plasma formation from the optical breakdown of a target on which laser pulses from a laser beam impinges, the improvement comprising:
the operating port positioned at the distal end of the needle,
the target having a wall mass which extends immediately proximal of the distal most portion of the operating port,
the needle having a wall which is unitary to define an aspirating channel having a smooth surface.

2. The surgical needle of claim 1 wherein:
said operating port is substantially on the first side of a plane longitudinally bisecting the surgical needle, and said target is substantially on the second side of said plane.

3. The surgical needle of claim 1 wherein: said operating port is substantially circular.

4. The surgical needle of claim 2 wherein: said operating port is substantially circular.

5. The surgical needle of claim 1 wherein:
said operating port has a central axis and said needle has a central axis, said central axis of said port and said central axis of said needle being at approximately 45 degrees to one another.

6. The surgical needle of claim 4 wherein:
said operating port has a central axis and said needle has a central axis, said central axis of said port and said central axis of said needle being at approximately 45 degrees to one another.

7. The surgical needle of claim 1 having a longitudinal channel through said needle, said channel being adapted to permit aspirating through said channel the tissue that is fractured at said operating port.

8. The surgical needle of claim 6 having a longitudinal channel through said needle, said channel being adapted to permit aspirating through said channel the tissue that is fractured at said operating port.

9. The surgical needle of claim 3 having an optical fiber for conveying the laser pulses, wherein:
the sole turbulent inducing structure in the aspirating channel proximal of said operating port and said target is the optical fiber.

10. The surgical needle of claim 8 having an optical fiber for conveying the laser pulses, wherein:
the sole turbulent inducing structure in the aspirating channel proximal of said operating port and said target is the optical fiber.

11. The surgical needle of claim 1 wherein: said target and said operating port extend over approximately the same distal longitudinal distance of the surgical needle.

12. The surgical needle of claim 10 wherein: said target and said operating port extend over approximately the same distal longitudinal distance of the surgical needle.
